(19) United States
(12) Patent Application Publication
(10) Pub. No.: US 2023/0364299 A1
(43) Pub. Date: Nov. 16, 2023

(54) FREEZE-DRIED COLLAGEN-SPONGE-TYPE COMPOSITION FOR BONE REGENERATION

(71) Applicant: Denhouse Co.,Ltd, Jeollabuk-do (KR)

(72) Inventor: Kyung-hwan Kwon, Jeollabuk-do (KR)

(73) Assignee: Denhouse Co.,Ltd, Jeollabuk-do (KR)

(21) Appl. No.: 18/251,010

(22) PCT Filed: Aug. 25, 2021
(86) PCT No.: PCT/KR2021/011352

§ 371 Date: Apr. 28, 2023

(30) Foreign Application Priority Data
Oct. 29, 2020 (KR) 10-2020-0142058

(51) Int. Cl. A61L 27/24; A61L 27/54; A61L 27/56; A61C 8/02

(52) U.S. Cl. CPC A61L 27/24; A61L 27/54; A61L 27/56; A61C 8/0006; A61L 2300/402; A61L 2300/45; A61L 2300/418; A61L 2430/02

(57) ABSTRACT

The present invention relates to a collagen sponge-type composition for bone regeneration characterized in that the composition is manufactured by homogenizing a hydrated collagen; and a mixed solution of the lidocaine, epinephrine and thrombin, and then lyophilizing same. The composition according to the present invention, which activates undifferentiated cells in bone marrow and also activates bone regeneration capacity in bone marrow through the activation and differentiation of osteoclasts and osteoblasts, may be a method for successful treatment of medication-related osteonecrosis of the jaw (MRONJ) through a bone marrow activation treatment method, and may develop the treatment of the fundamental causes of MRONJ through a bone marrow activation treatment method using various growth factors for bone marrow activation and bone marrow activation factors such as cytokines and completely regenerate jaw bone conditions aesthetically and functionally. Accordingly, the composition according to the present invention may be useful in the treatment of various diseases caused by bone remodeling failure.

### TECHNICAL FIELD

The present invention relates to a freeze-dried collagen-sponge-type composition for bone regeneration capable of activating undifferentiated cells in bone marrow and also activating bone regeneration capacity in bone marrow through the activation and differentiation of osteoclasts and osteoblasts.

### BACKGROUND ART

In the dental field, innovative treatment methods have constantly developed as clinical knowledge on bone has constantly developed and tissue engineering in the bone regeneration field along with molecular biology has developed. However, recently, therapeutic agents for osteoporosis, anti-angiogenic drugs, steroid immunosuppressive agents, therapeutic agents for hyperlipidemia, etc., are widely used for long term, which leads to various intractable diseases such as osteonecrosis or osteomyelitis. Thus, it is not easy to conduct treatment in practice.

The first discovery of osteonecrosis caused by alendronate injections in 2003 by Marx (Marx RE (September 2003). “Pamidronate (Aredia) and zoledronate (Zometa) induced avascular necrosis of the jaws: a growing epidemic” J. Oral Maxillofac. Surg. 61 (9): 1115-7), and bisphosphonate (the drug for treatment of osteoporosis)—related osteonecrosis was also discovered in Korea after 2005. It was misidentified as a type of osteomyelitis and treated by a method for treating osteomyelitis. However, osteomyelitis or osteonecrosis symptoms persisted, and side effects of invading into larger area and complications occurred.

Case Presentation 1-1 of a patient with multiple myeloma misidentified as osteomyelitis in 2005, who was administered with an injection of Actonel (a bisphosphonate): Osteonecrosis in the jaw or local necrosis of bone tissue were rare potential complications for patients with cancer who received radiotherapy or chemotherapy, or received treatment for patients with tumor or infectious embolism. In 2003, there was a report that a risk of osteonecrosis increased in patients who were treated in combination with bisphosphonate intravenous administration.

Matrix metalloproteinase 2 may be a candidate gene for bisphosphonate-related osteonecrosis in the jaw, and it is the only gene involved in bone abnormalities and atrial fibrillation. Thus, there is a report that it may cause another side effect of bisphosphonates.

In 2005, the US Food and Drug Administration (FDA) released a broader drug class warning of osteonecrosis complications for all bisphosphonates.

Since 2007, through multi-instrumental examination by Marx on patients who took a broad range of bisphosphonates, it was reported that extensive osteonecrosis was being occurred. Also, there have been reports in Korea since 2007 announcing that osteonecrosis has occurred. However, the studies on principle or mechanism of medication-related osteonecrosis were not sufficient. In particular, bone biology has developed mostly regarding the studies on osteoblasts.

The recently revealed cause of osteonecrosis was recognized as phenomenon associated with malfunction of osteoclasts, thereby resulting in hindrance of the chemical migration or differentiation of osteoblasts with bone regeneration capacity. As such, it was known that the activity of osteoclasts, rather than an increase in the number of osteoblasts, plays a crucial role in bone regeneration.

Bone cells include osteoblasts, osteocytes, and osteoclasts. In the fields of physiology or biology studies on bone since 2000, attention has focused on an increase in the number of osteoblasts and osteocytes using stem cells or undifferentiated cells and an increase in the production rate of bone.

In particular, as for bone grafting for dental implants, attention has focused on bone materials that can increase osteocytes. However, the main attention regarding these bone materials has focused on osteoconduction. As for the treatment of bone for osteoporosis or rheumatoid arthritis, the studies were focused on inhibition of the number or action of osteoclasts, and it was vaguely agreed that osteoporosis or rheumatoid arthritis could be treated by such treatment methods.

Since 2010, both researchers who study bone biology, bone grafting, or bone curing and clinicians have proved that increasing or decreasing the number of osteocytes or osteoclasts is not the way of improving the success rate of bone curing or bone grafting.

With the facts alone that side effects of bisphosphonates widely used to treat osteoporosis broadly increase, and that various drugs, steroids, and nonopioid analgesics used to treat rheumatoid arthritis cause side effects of inducing osteonecrosis and reducing bone immunity, a need for development of novel studies are being spread.

Bone is a composite tissue of proteins and minerals and is continuously remodeling itself. Osteoclasts, bone absorbing materials and osteoblasts which form bone play a role in bone growth, damage treatment, regulation in the metabolism of calcium and phosphate and absorb the bone during remodeling. They are recognized as basic multicellular units for forming the bone.

Osteoclasts are large multinucleated cells distinguished from bone marrow precursors, controlled by macrophage colony-stimulating factor (MCSF) (cytokines provided by osteocytes) and receptor activator of NF NF-κB ligand (RANKL). Osteoclasts hydrolyze proteinase (e.g., cathepsin K) and acid, and digest and dissolve organic and inorganic components of the bone matrix.

When the coupling between osteoclasts and osteoblasts is broken, the delicate balance between the bone resorption activities and the bone formation activities is lost resulting in bone diseases. Such phenomena are reported as side effects of bisphosphonates when used as the therapeutic agent for patients with osteoporosis. Actonel, Bonviva, etc., which are bisphosphonate drugs initially based on the mechanism of treating osteoporosis by reducing reabsorption mechanism to induce bone loss, destroyed the coupling network relationship between osteoclasts and osteoblasts and disabled the mechanism for bone regeneration.

Osteonecrosis of the jaw (ONJ) is an oral disease accompanying the exposure of the jaw bone. Some patients may experience no symptoms, but ONJ generally involves pain and pus discharge.

This symptom may occur naturally, or after extraction of a tooth, wounds, or radiotherapy to the head and neck (referred to as osteoradionecrosis).

ONJ may be actually bone infection (osteomyelitis), not osteonecrosis. ONJ is observed in people to whom recently a high dose of bisphosphonates is administered intravenously, in particular people who suffered from cancer or received oral surgery while being administered with this drug.

There were reports that ONJ is not associated with the ordinary use of bisphosphonates orally administered for treatment of osteoporosis. However, recently it was informed that in the case of long-term use of the drug for 3 years or more, osteonecrosis occurs. It was revealed that such symptoms are caused by malfunction of osteoclasts.

In Korea, it has been reported that the prevalence rate of bisphosphonate-related osteonecrosis of the jaw (BRONJ), the representative disease of medication-related osteonecrosis of the jaw, is about between 2 and 10% in patients who were orally administered with bisphosphonates for 3 years or more or with an injection thereof for 1 year or more. It is known that the administration of drugs in combination amplifies the rate.

In particular, it was reported that when administering steroids used to treat arthritis and bisphosphonates in combination or when simultaneously administering the therapeutic agents for hyperlipidemia and bisphosphonates, the prevalence rate of osteonecrosis further increased. Thus, it is most important for dental clinic to analyze whether drugs are administered and to assay the components thereof.

Bones are always regenerated by osteoclasia and osteogenesis. Bones providing support for the human body act as reserves of minerals such as calcium. When renewing bones, old bones should be broken down by osteoclasts.

When osteoclasts do not function properly, interleukins such as IL-1 and IL-6 are not secreted, and the cells do not play a role in the differentiation and regeneration of osteoblasts of interleukins, failing to bone regeneration. Further, osteoblasts strongly express receptor activator of nuclear factor kappa-B ligand (RANKL) necessary for raising osteoclasts. It could be confirmed from experiments that in mice having osteoblasts with RANKL removed, osteoclasts do not grow and severe marble bone diseases occur.

### PRIOR ART LITERATURE

### Patent Literature

(Patent Document 1) Korean Patent No. 10-0894265

(Patent Document 2) Korean Laid-Open Patent Publication No. 10-201-0092056

### DETAILED DESCRIPTION

### Technical Objective

Therefore, the objective that the present invention intends to solve is to provide a lyophilized collagen sponge-type composition for bone regeneration capable of activating undifferentiated cells in bone marrow and also activating bone regeneration capacity in bone marrow through the activation and differentiation of osteoclasts and osteoblasts.

### MEANS FOR SOLVING TECHNICAL OBJECTIVE

In order to solve the above objective, the present invention provides a collagen sponge-type composition for bone regeneration, characterized in that the composition is manufactured by homogenizing a hydrated collagen; and a mixed solution of the lidocaine, epinephrine and thrombin, and then lyophilizing same.

The collagen sponge-type composition for bone regeneration according to the present invention as described above is characterized in that the content of collagen in the hydrated collagen is 3 to 6% (w/v).

The collagen sponge-type composition for bone regeneration according to the present invention as described above is characterized in that the content of lidocaine in the mixed solution is 1 to 3% (w/v).

The collagen sponge-type composition for bone regeneration according to the present invention as described above is characterized in that the content of epinephrine in the mixed solution is 1: 50,000 to 200,000 (v/v) relative to lidocaine.

The collagen sponge-type composition for bone regeneration according to the present invention as described above is characterized in that the content of thrombin in the mixed solution is 1 to 3% (w/v).

The collagen sponge-type composition for bone regeneration according to the present invention as described above is characterized in that the ratio of the hydrated collagen and the mixed solution of the lidocaine, epinephrine and thrombin is 1: 0.3 to 1.7 (w/w).

### WORKING EFFECT OF THE INVENTION

The composition according to the present invention, which activates undifferentiated cells in bone marrow and also activates bone regeneration capacity in bone marrow through the activation and differentiation of osteoclasts and osteoblasts, may be a method for successful treatment of medication-related osteonecrosis of the jaw (MRONJ) through a bone marrow activation treatment method, and may develop the treatment of the fundamental causes of MRONJ through a bone marrow activation treatment method using various growth factors for bone marrow activation and bone marrow activation factors such as cytokines and completely regenerate jaw bone conditions aesthetically and functionally. Accordingly, the composition according to the present invention may be useful in the treatment of various diseases caused by bone remodeling failure.

### BRIEF DESCRIPTION OF THE DRAWINGS

FIGS. 1a-1c are pictures about a lyophilizing process.

FIGS. 2a-2e are pictures of applying the composition according to the present invention to the right molar region of the upper jaw.

FIGS. 3a-3j are pictures of applying the composition according to the present invention to the left front tooth region of the upper jaw.

### BEST MODE FOR CARRYING OUT THE INVENTION

Hereinafter, the present invention will be described in detail.

The present invention provides a collagen sponge-type composition for bone regeneration characterized in that the composition is manufactured by homogenizing a hydrated collagen; and a mixed solution of the lidocaine, epinephrine and thrombin, and then lyophilizing same.

The thrombin promotes proliferation of osteoblasts while inhibiting apoptosis thereof via activation of protease-activated receptor-1 (PAR-1). The promotion serves as osteoprotegerin (OPG) which inhibits collagen secretion and RANKL using osteoblasts. Thrombin plays a role at the initial stage of bone regeneration, allowing the differentiation of osteoblasts and the inhibition of osteoclasts at the initial stage. When lidocaine-thrombin-collagen, which is a bone marrow activation scaffold, is adequately mixed, pain or inflammation induction at the initial stage is reduced and vascularization is induced, and the vascularization positively stimulates the migration of undifferentiated stem cells.

Lidocaine is an amide-type local anesthetic synthesized by Swedish chemists Nils Lofgren and Bengt Lundqvist in 1943. Lidocaine is slightly inferior in terms of action or duration to tetracaine known as the strongest local anesthetic. However, lidocaine is less toxic than tetracaine and has sufficient efficacy. Thus, lidocaine is a local anesthetic generally safe for use in local anesthesia. It may be used with the addition of epinephrine to prolong its effects as an anesthetic in a small amount and to suppress bleeding by hemostatic action during surgery, by contracting blood vessels around the anesthetized site. It has high fat-soluble protein binding capacity and prolonged duration, which allows good dissolution with rhBMP-2 and collagen proteins, and thus it may also be used as a solvent.

It may induce the formation into a sustained release capsule through ionic bonding in constituting a sustained-release type scaffold along with the effect of inhibiting initial inflammatory response caused by thrombin. Using lidocaine as a solvent provides various advantages in clinical uses.

First, it has the effect of significantly reducing the occurrence of pain caused by inflammatory conditions. Second, it has the effect of slowly releasing the drug which is dissolved in a lidocaine solvent.

In addition, as epinephrine-containing lidocaine is used, it may reduce the amount absorbed into the neighboring tissues during the maximum length of time of one and a half hours in which the drug is released.

Third, it neutralizes acidic collagen solution, which leads to a change into a hard lump in which the solidity of crystals overall increases. With these three roles and advantages, the function of a scaffold of the LT collagen can be provided.

When a mixture of thrombin and lidocaine is implanted with the components of the bone marrow activation of the present invention within the initial 2 hours, hemorrhage in bone marrow is decreased and minimum edema is exhibited, which indicate the bone curing.

In addition, in view that the necessity for developments of bone graft using thrombin are continuously emerging, the present invention is useful in that it can utilize the initial process for bony tissues and various bone regeneration capacities of thrombin.

The collagen sponge-type composition for bone regeneration according to the present invention as described above is characterized in that the content of collagen in the hydrated collagen is 3 to 6% (w/v).

The collagen sponge-type composition for bone regeneration according to the present invention as described above is characterized in that the content of lidocaine in the mixed solution is 1 to 3% (w/v).

The collagen sponge-type composition for bone regeneration according to the present invention as described above is characterized in that the content of epinephrine in the mixed solution is 1: 50,000 to 200,000 (v/v) relative to lidocaine.

The collagen sponge-type composition for bone regeneration according to the present invention as described above is characterized in that the content of thrombin in the mixed solution is 1 to 3% (w/v).

The collagen sponge-type composition for bone regeneration according to the present invention as described above is characterized in that the ratio of the hydrated collagen and the mixed solution of the lidocaine, epinephrine and thrombin is 1: 0.3 to 1.7 (w/w).

The above-described content ratios are determined based on various experiments and experience of the inventors of the present invention, and are the optimal ratios for achieving the best effect of the present invention.

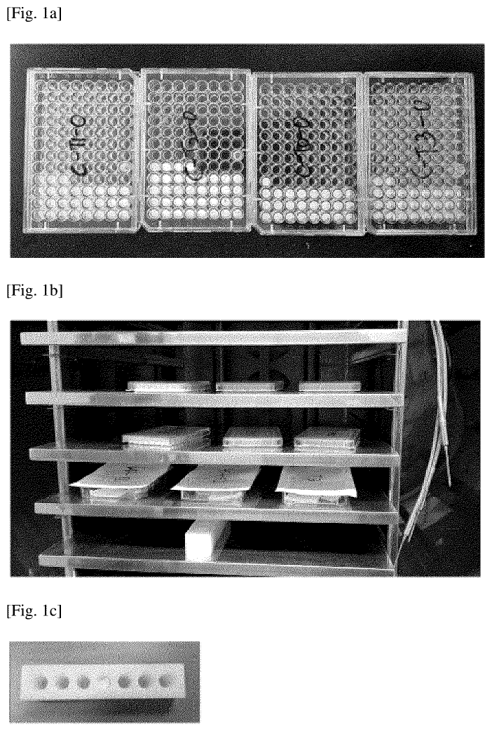

Also, the homogenizing and lyophilizing methods of the hydrated collagen and the mixed solution of the lidocaine, epinephrine and thrombin may be used without particular limitation as long as they are widely known in the art to which the present invention belongs. The lyophilizing process is as shown in FIGS. 1a-1c.

The composition according to the present invention, which activates undifferentiated cells in bone marrow and also activates bone regeneration capacity in bone marrow through the activation and differentiation of osteoclasts and osteoblasts, may be a method for successful treatment of medication-related osteonecrosis of the jaw (MRONJ) through a bone marrow activation treatment method, and may develop the treatment of the fundamental causes of MRONJ through a bone marrow activation treatment method using various growth factors for bone marrow activation and bone marrow activation factors such as cytokines and completely regenerate jaw bone conditions aesthetically and functionally. This will be described in detail with the following examples.

### EXAMPLES

### Patients and Treatment

### Manufacturing Process

1.5 ml of a collagen-thrombin-lidocaine complex mixture (OSSCORE LMT collagen scaffold, Lidocaine-Maltodextrin-Thrombin collagen) was made by mixing thrombin (thrombin lyophilized powder 5000 unit—Reyon Phamaceutical Co., Ltd., pharmaceutical product extracted from bovine plasma, imported from Germany), lidocaine (using 0.5 ml of 1.8 ml containing epinephrine 1:100,000), and 1 cc of 6% hydrated collagen (OSSCORE Denhouse). The complex mixture was filled in a conical mold and lyophilized to produce a collagen sponge-type composition for bone regeneration according to the present invention.

### Methods for Using the Composition

Through the cortical perforations made in the buccal cortical bone and the lingual cortical bone, the composition was filled in the bone marrow, while allowing the blood to be smoothly supplied to the bone marrow. After that, the incision site was sutured using a continuous locking suture and an interrupted suture in combination, so that the bone marrow site is not exposed. The suture was made using Vicryl 4-0 absorbable suture material and maintained for at least 2 weeks.

The patient was administered with penicillin antibiotics and clindamycin antibiotics in combination, and the administration was retained for 7 days after surgery. The suture material of the loose portion was removed after 2 weeks or more, and the patient was allowed to take soft foods until the mucous membrane was completely sutured and not to take out his dentures. The patient was examined persistently at 1 month, 3 months, 6 months and 12 months, and the disease has not recurred for 5 years.

### Pictures of the Treatment Process

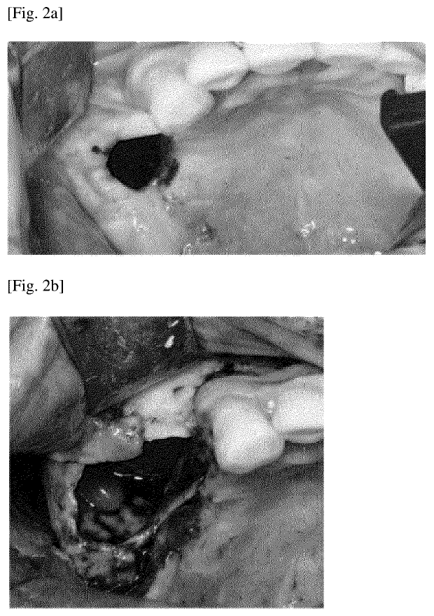

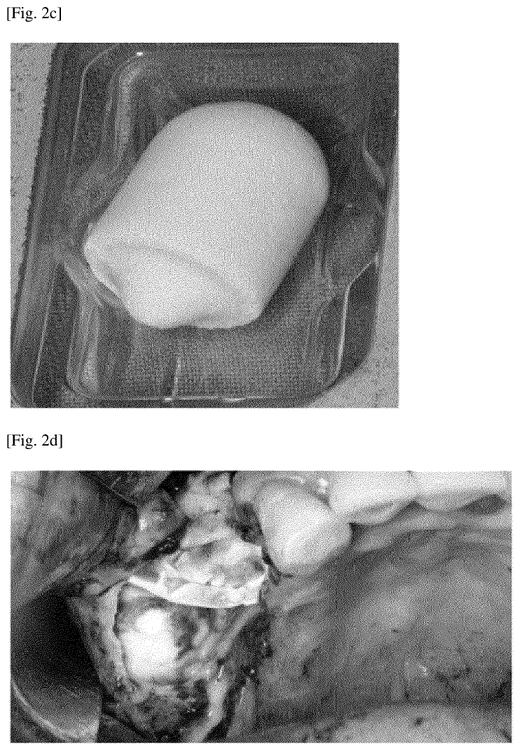

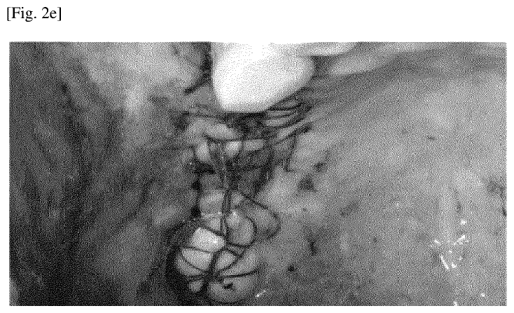

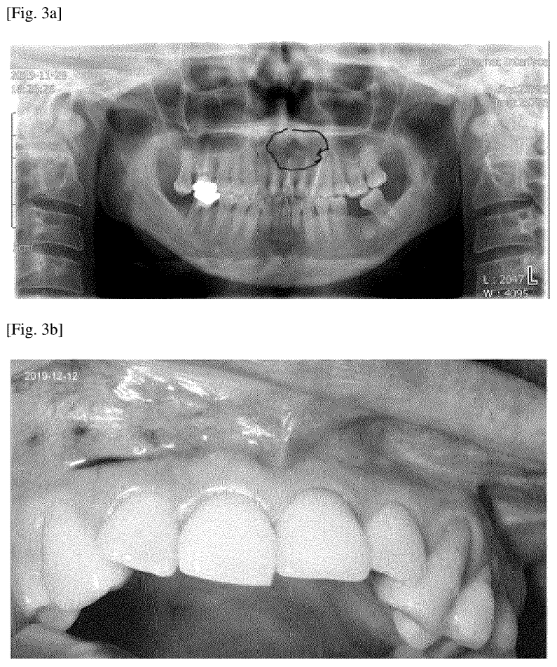

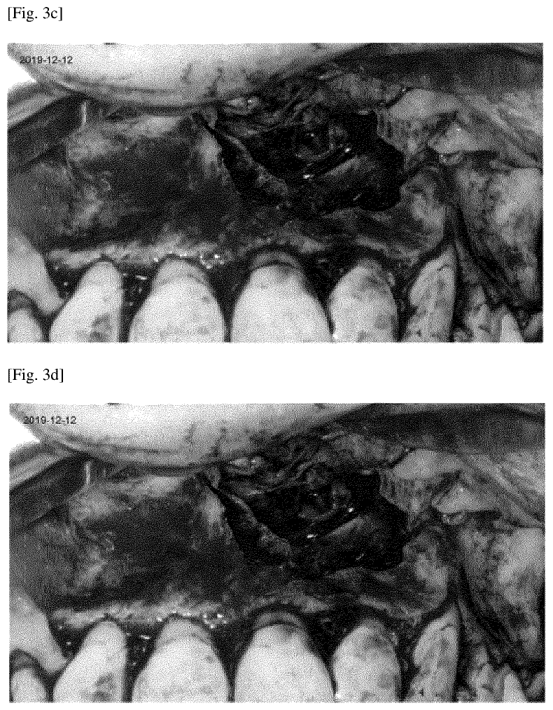

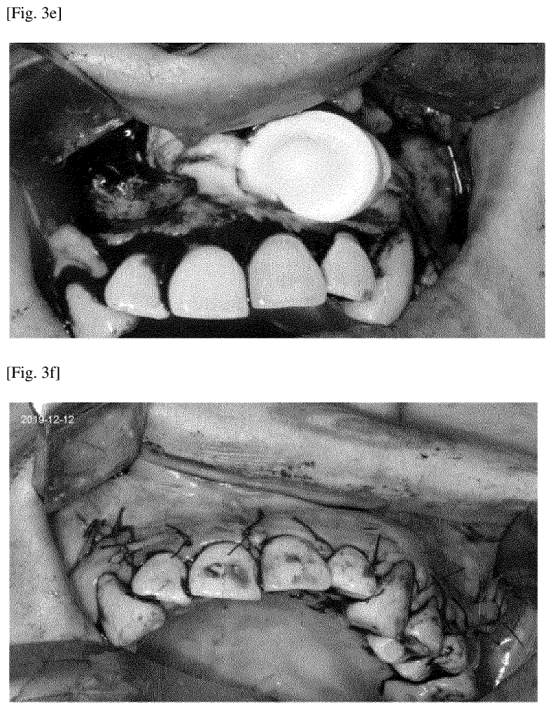

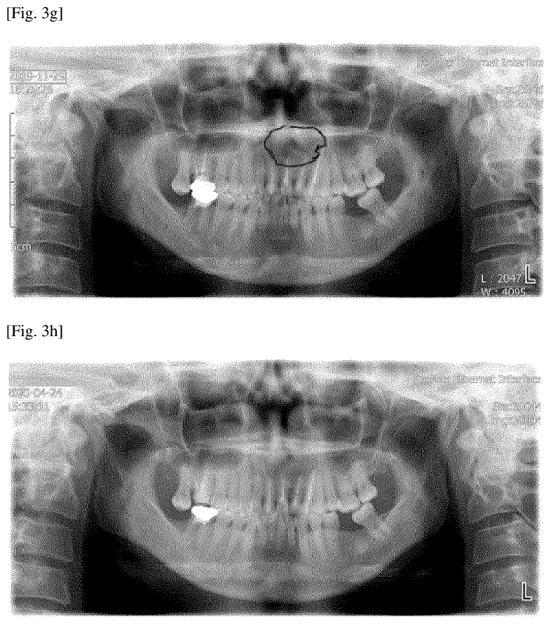

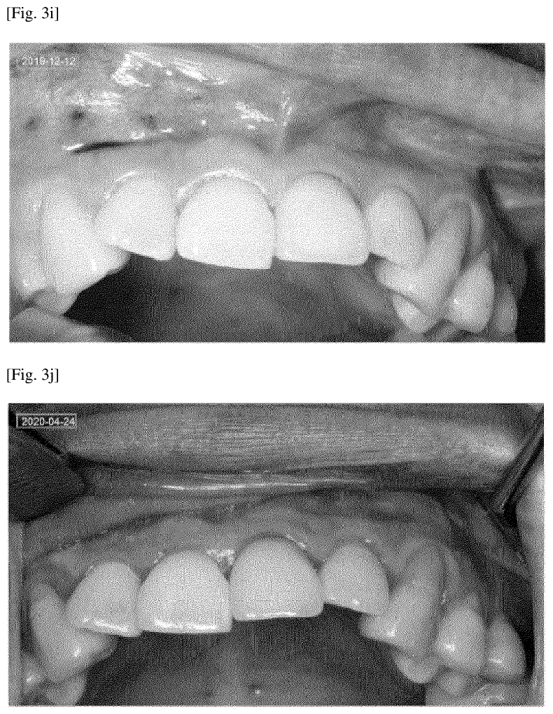

FIGS. 2a-2e are the pictures of applying the composition according to the present invention to the right molar region of the upper jaw, and FIGS. 3a-3j are pictures of applying the composition according to the present invention to the left front tooth region of the upper jaw.

The drawings for other patients who underwent similar treatment processes are provided in FIGS. 8a, 8b and 8c. From these examples, the effect of the composition according to the present invention could also be confirmed.

What is claimed is:

1. A collagen sponge-type composition for bone regeneration characterized in that the composition is manufactured by homogenizing a hydrated collagen; and a mixed solution of the lidocaine, epinephrine and thrombin, and then lyophilizing same.

2. The collagen sponge-type composition for bone regeneration of claim 1,
wherein the content of collagen in the hydrated collagen is 3 to 6% (w/v).

3. The collagen sponge-type composition for bone regeneration of claim 1,
wherein the content of lidocaine in the mixed solution is 1 to 3% (w/v).

4. The collagen sponge-type composition for bone regeneration of claim 1,
wherein the content of epinephrine in the mixed solution is 1: 50,000 to 200,000 (v/v) relative to lidocaine.

5. The collagen sponge-type composition for bone regeneration of claim 1,
wherein the content of thrombin in the mixed solution is 1 to 3% (w/v).

6. The collagen sponge-type composition for bone regeneration of claim 1,
wherein the ratio of the hydrated collagen and the mixed solution of the lidocaine, epinephrine, and thrombin is 1: 0.3 to 1.7 (w/w).
